(19) United States
(12) Patent Application Publication
(10) Pub. No.: US 2013/0052214 A1
(43) Pub. Date: Feb. 28, 2013

(54) IDENTIFICATION OF A PEPTIDE(S) THAT INDUCES AUTOIMMUNE ANTERIOR UVEITIS

(71) Applicants: Nalini Bora, Little Rock, AR (US); Purushottam Jha, Little Rock, AR (US); Puran Bora, Little Rock, AR (US)

(72) Inventors: Nalini Bora, Little Rock, AR (US); Purushottam Jha, Little Rock, AR (US); Puran Bora, Little Rock, AR (US)

(73) Assignee: BOARD OF TRUSTEES OF THE UNIVERSITY OF ARKANSAS, Little Rock, AR (US)

(21) Appl. No.: 13/595,402

(22) Filed: Aug. 27, 2012

Related U.S. Application Data
(60) Provisional application No. 61/527,427, filed on Aug. 25, 2011.

(51) Int. Cl. A61K 39/00; A61P 37/02; A61P 27/02; C07K 7/08; C07K 14/435

(52) U.S. Cl. 424/185.1; 530/326; 530/325; 530/324

(57) ABSTRACT

The present invention provides an immunogenic composition effective for inducing, treating, reducing the incidence of or severity of clinical signs associated with autoimmune anterior uveitis.

### CROSS REFERENCE TO RELATED APPLICATIONS

This application claims the priority of U.S. provisional application No. 61/527,427, filed Aug. 25, 2011 which is hereby incorporated by reference in its entirety.

### FIELD OF THE INVENTION

The present invention provides an immunogenic composition effective for inducing, treating, reducing the incidence of or reducing the severity of clinical signs associated with autoimmune anterior uveitis.

### BACKGROUND

Anterior uveitis is an inflammation of the middle layer of the eye, which includes the iris (colored part of the eye) and adjacent tissue, known as the ciliary body. If untreated, it can cause permanent damage and loss of vision from the development of glaucoma, cataract or retinal edema. In the majority of cases, anterior uveitis is an autoimmune disorder. Most people with autoimmune anterior uveitis actually don't have a systemic autoimmune disease, but rather have a localized autoimmune dysfunction involving the uveal tract. Finding the antigen that triggers the autoimmune response will aid in both research into the etiology of autoimmune anterior uveitis, and in methods of inducing tolerance to the autoantigen.

### SUMMARY OF THE INVENTION

One aspect of the present invention encompasses an immunogenic composition. The composition comprises a truncated peptide consisting of a peptide with at least 90% homology to at least 15 contiguous amino acids selected from amino acids 101 to 150 of SEQ ID NO:3.

Another aspect of the present invention encompasses a method of inducing experimental autoimmune anterior uveitis in a subject. The method comprises administering an immunogenic composition to a subject. The composition comprises a truncated peptide consisting of a peptide with at least 90% homology to at least 15 contiguous amino acids selected from amino acids 101 to 150 of SEQ ID NO:3.

Yet another aspect of the present invention encompasses a method of treating anterior uveitis in a subject. The method comprises the step of administering an immunogenic composition to a subject in need thereof. The composition comprises a truncated peptide consisting of a peptide with at least 90% homology to at least 15 contiguous amino acids selected from amino acids 101 to 150 of SEQ ID NO:3.

### REFERENCE TO COLOR FIGURES

The application file contains at least one photograph executed in color. Copies of this patent application will be provided by the Office upon request and payment of the necessary fee.

### DESCRIPTION OF THE DRAWING FIGURES

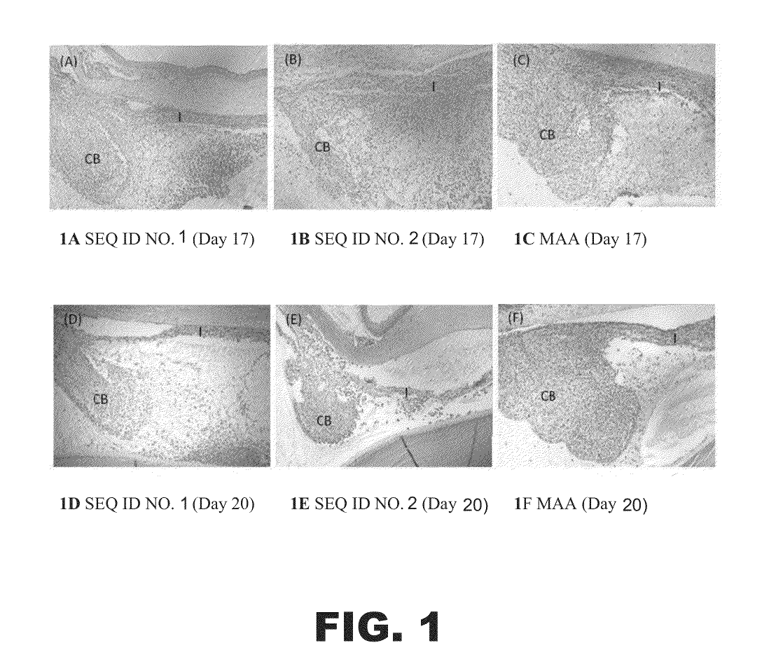

FIG. 1A depicts a color photograph of representative histologic sections of rat eye stained with H&E depicting the induction of EAAU by MAA derived peptide SEQ ID NO. 1 in an animal sacrificed at day 17, post immunization;

FIG. 1B depicts a color photograph of representative histologic sections of rat eye stained with H&E depicting the induction of EAAU by MAA derived peptide SEQ ID NO. 2 in an animal sacrificed at day 17, post immunization;

FIG. 1C depicts a color photograph of representative histologic sections of rat eye stained with H&E depicting the induction of EAAU by bovine MAA in an animal sacrificed at day 17, post immunization;

FIG. 1D depicts a color photograph of representative histologic sections of rat eye stained with H&E depicting the induction of EAAU by MAA derived peptide SEQ ID NO. 1 in an animal sacrificed at day 20, post immunization;

FIG. 1E depicts a color photograph of representative histologic sections of rat eye stained with H&E depicting the induction of EAAU by MAA derived peptide SEQ ID NO. 2 in an animal sacrificed at day 20, post immunization; and

FIG. 1F depicts a color photograph of representative histologic sections of rat eye stained with H&E depicting the induction of EAAU by bovine MAA.

### DETAILED DESCRIPTION

The present invention overcomes the problems inherent in the prior art and provides a distinct advance in the state of the art. Generally, the present invention provides an immunogenic composition effective for inducing, treating, reducing the incidence of, or reducing the severity of clinical signs associated with autoimmune anterior uveitis.

### I. Immunogenic Composition

In preferred forms the immunogenic composition comprises a truncated peptide derived from or based on the sequence of Melanin Associated Antigen (MAA). Even more preferably, the truncated peptide of the present invention is derived from or based on a 22 kDa fragment of type I collagen±2 chain (CI−±2 (22 kDa) with SEQ ID NO. 3 (IGPAGP PGPPGLRGNP GSRGLPGADG RAGVMGPAGS RGATGPAGVR GPNGDSGRPG EPGLMGPRGF PGSPGNIGPA GKEGPVGLPG IDGRPGPIGP AGARGEPGNI GFPGPKGPSG DPGKAGEKGH AGLAGARGAP GPDGNNGAQG PPGLQGVQGG KGEQGPAGPP GFQGLPGPAG TAGEAGKPGE RGIPGEFGLP GPAG) derived from bovine as well as rat iris and ciliary body (“CB”) and consisting of about 200 amino acids. Still more preferably, the truncated peptide of the present invention includes a peptide having at least 90%, more preferably at least 93%, still more preferably at least 95%, even more preferably at least 97%, still more preferably at least 99%, and most preferably 100% sequence homology or sequence identity sequence with a peptide having 15 to 40, more preferably 17 to 35, still more preferably 20 to 30, even more preferably 22 to 28, and most preferably about 25 consecutive amino acids from MAA. Preferably, the consecutive amino acids are from the 22 kDa fragment discussed above.

In certain embodiments, a truncated peptide of the invention consists of a peptide with at least 90% homology to at least 15 contiguous amino acids selected from amino acids 101 to 150 of SEQ ID NO:3. For instance, a truncated peptide of the invention may consist of a peptide with at least 90% homology to at least 15, 20, 25, 30, 35, 40, 45, or 50 contiguous amino acids selected from amino acids 101 to 150 of SEQ ID NO:3. In alternative embodiments, a truncated peptide of the invention may consist of a peptide with at least 90%, 91%, 92%, 93%, 94%, 95%, 96%, 97%, 98%, or 99% homology to at least 15, 20, 25, 30, 35, 40, 45, or 50 contiguous amino acids selected from amino acids 101 to 150 of SEQ ID NO:3. In one embodiment, a truncated peptide of the invention may consist of a peptide with 100% homology to at least 15, 20, 25, 30, 35, 40, 45, or 50 contiguous amino acids selected from amino acids 101 to 150 of SEQ ID NO:3. For example, a truncated peptide of the invention may consist of a peptide with 100% homology to at least 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 contiguous amino acids selected from amino acids 101 to 150 of SEQ ID NO:3.

In some embodiments, the truncated peptide consists of a peptide having at least 90%, more preferably at least 93%, still more preferably at least 95%, even more preferably at least 97%, still more preferably at least 99%, and most preferably 100% sequence homology or sequence identity with SEQ ID NO. 1 (GEPGNIGFPGPKGPSGDPGKAGEKG) or SEQ ID NO. 2 (HAGLAGARGAPGPDGNNGAQGPPGL). For example, in one embodiment, a truncated peptide of the invention consists of SEQ ID NO:1. In another embodiment, a truncated peptide of the invention consists of SEQ ID NO:2.

In yet other embodiments, a truncated peptide of the invention consists of a peptide having the sequence of SEQ ID NO:1 or 2 but for one or two conservative amino acid substitutions. In another embodiment, a truncated peptide of the invention consists of a peptide with 100% homology to at least 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 contiguous amino acids selected from amino acids 101 to 150 of SEQ ID NO:3, but for one, two, three, or four conservative amino acid substitutions.

In some forms of the invention, the immunogenic composition further comprises a pharmaceutically-acceptable carrier, preferably selected from the group consisting of solvents, dispersion media, coatings, adjuvants, stabilizing agents, diluents, preservatives, antibacterial and antifungal agents, isotonic agents, adsorption delaying agents, and combinations thereof. One particularly preferred composition includes saline, preferably phosphate buffered saline. Another particularly preferred composition includes Freund's complete adjuvant. Yet another preferred composition includes both phosphate buffered saline and Freund's complete adjuvant.

### II. Methods of Inducing EAAU

Another aspect of the present invention comprises a method of inducing experimental autoimmune anterior uveitis (EAAU). Methods for inducing EAAU in animal models would be useful for studying the immunopathogenesis of uveitis or identifying and studying the autoantigen for anterior uveitis. Generally, the method comprises administering an immunogenic composition of the present invention, as described above, to a subject. The immunogenic composition may be administered in any suitable conventional manner including orally, nasally, via an injection (including intravenously, intramuscularly, and subcutaneously) intraocularly, topically, and combinations thereof. Of these, intraocularly and via injection are preferred. In preferred forms, the immunogenic composition administered to the subject further comprises a pharmaceutically-acceptable carrier selected from the group consisting of solvents, dispersion media, coatings, adjuvants, stabilizing agents, diluents, preservatives, antibacterial and antifungal agents, isotonic agents, adsorption delaying agents, and combinations thereof. Preferred subjects for this method may include rodents, such as rats, primates, and humans. The administration step can be done a single time or repeated once or multiple times, depending upon the desired effect. The amount of peptide administered can also vary depending on the desired effect. Generally, to induce EAAU, 10 μg to 1500 μg of said peptide per kg of subject is present in each administration of the immunogenic composition. More preferably, the amount ranges from 25 μg to 1000 μg, still more preferably 50 μg to 750 μg, even more preferably 65 μg to 600 μg, still more preferably 70 μg to 500 μg, and most preferably 75 μg to 400 μg of said peptide per kg of subject.

### III. Methods of Treatment

In another aspect of the present invention, a method for the treatment or prophylaxis of anterior uveitis is provided such that the severity of or incidence of clinical signs of anterior uveitis are reduced in comparison to a subject not undergoing or experiencing the described method. In terms of prophylaxis of anterior uveitis, prophylaxis will include any reduction in the severity of clinical signs of anterior uveitis in comparison to a subject not undergoing or experiencing the described method. Preferably, the reduction in severity or incidence of clinical signs from treatment or prophylaxis will be at least 10%, more preferably at least 25%, still more preferably at least 50%, even more preferably at least 60%, still more preferably at least 70%, even more preferably at least 80%, still more preferably at least 90%, even more preferably at least 95%, and most preferably 100%, in comparison to subjects not receiving the treatment or prophylaxis. Generally, the method comprises administering an immunogenic composition of the present invention, as described above, to a subject. Preferably, the subject will already have anterior uveitis if undergoing treatment, and will not have anterior uveitis (or be between bouts of recurring anterior uveitis) if the method is being used prophylactically. The immunogenic composition can be administered in any suitable conventional manner including orally, nasally, via an injection (including intravenously, intramuscularly, and subcutaneously) intraocularly, topically, and combinations thereof. Of these, intraocularly and via injection are preferred. In preferred forms, the immunogenic composition administered to the subject further comprises a pharmaceutically-acceptable carrier selected from the group consisting of solvents, dispersion media, coatings, adjuvants, stabilizing agents, diluents, preservatives, antibacterial and antifungal agents, isotonic agents, adsorption delaying agents, and combinations thereof. Preferred subjects for this method may include rodents, such as rats, primates, and humans. The administration step can be done a single time or repeated once or multiple times, depending upon the desired effect. The amount of peptide administered can also vary depending on the desired effect. Generally, to treat EAAU, 10 μg to 1500 μg of said peptide per kg of subject is present in each administration of the immunogenic composition. More preferably, the amount ranges from 25 μg to 1000 μg, still more preferably 50 μg to 750 μg, even more preferably 65 μg to 600 μg, still more preferably 70 μg to 500 μg, and most preferably 75 μg to 400 μg of said peptide per kg of subject. Generally, to use the immunogenic composition of the invention prophylactically, 10 μg to 1500 μg of the peptide per kg of subject is present in each administration of the immunogenic composition. More preferably, the amount ranges from 15 μg to 1000 μg, still more preferably 20 μg to 750 μg, even more preferably 25 μg to 500 μg, still more preferably 30 μg to 250 μg, and most preferably 35 μg to 150 μg of said peptide per kg of subject. In preferred forms of using the immunogenic composition prophylactically, or for treatment, an amount of the immunogenic composition is repeatedly administered in order to induce immune tolerance of the peptide in the subject. Successful induction of immune tolerance results in a reduction in the severity of or incidence of clinical signs of anterior uveitis. This includes a reduction in the number of episodes of recurring anterior uveitis.

The methods described above would also be useful in identifying specific antigen or uveitogenic T cells or T regulatory cells in subjects, including patients with anterior uveitis. This would be done by obtaining samples potentially containing such immune components and analyzing such samples for the immune components contained therein. Once identified, the production or regulation of these components could be monitored during the course of anterior uveitis.

### DEFINITIONS

As used herein, a “conservative amino acid substitution” is one in which the amino acid residue is replaced with an amino acid residue having a similar side chain. Families of amino acid residues having similar side chains have been defined in the art. These families include amino acids with basic side chains (e.g. lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The resulting peptide comprising the substitution should have similar characteristics or properties including size, hydrophobicity, etc., such that the overall functionality of the peptide does not significantly change.

For purposes of the present invention, the term “truncated peptide” refers to a peptide that is shorter than a full length reference peptide. For example, the preferred truncated peptide of the present invention is a shorter version of the MAA amino acid sequence.

The phrase “derived from or based on” when referring to the peptide of the present invention refers to the source material upon which the peptide sequences of the present invention are based. For example, when a peptide is “derived from” another amino acid sequence, it can be an isolated and/or purified form of the peptide. Similarly, when a peptide is “based on” another amino acid sequence, the other amino acid sequence is used as a guide upon which to construct a peptide sequence. In terms of the present invention, the sequence of MAA, or portions thereof can be purified and fragmented to provide several smaller sequences that were “derived from” the original MAA sequence. If these smaller sequences were then synthesized, the synthesized sequences would be “based on” the original MAA sequence.

“Sequence Identity” as it is known in the art refers to a relationship between two or more polypeptide sequences or two or more polynucleotide sequences, namely a reference sequence and a given sequence to be compared with the reference sequence. Sequence identity is determined by comparing the given sequence to the reference sequence after the sequences have been optimally aligned to produce the highest degree of sequence similarity, as determined by the match between strings of such sequences. Upon such alignment, sequence identity is ascertained on a position-by-position basis, e.g., the sequences are “identical” at a particular position if at that position, the nucleotides or amino acid residues are identical. The total number of such position identities is then divided by the total number of nucleotides or residues in the reference sequence to give % sequence identity. Sequence identity can be readily calculated by known methods, including but not limited to, those described in Computational Molecular Biology, Lesk, A. N., ed., Oxford University Press, New York (1988), Biocomputing: Informatics and Genome Projects, Smith, D. W., ed., Academic Press, New York (1993); Computer Analysis of Sequence Data, Part I, Griffin, A. M., and Griffin, H. G., eds., Humana Press, New Jersey (1994); Sequence Analysis in Molecular Biology, von Heinge, G., Academic Press (1987); Sequence Analysis Primer, Gribskov, M. and Devereux, J., eds., M. Stockton Press, New York (1991); and Carillo, H., and Lipman, D., SIAM J. Applied Math., 48: 1073 (1988), the teachings of which are incorporated herein by reference. Preferred methods to determine the sequence identity are designed to give the largest match between the sequences tested. Methods to determine sequence identity are codified in publicly available computer programs which determine sequence identity between given sequences. Examples of such programs include, but are not limited to, the GCG program package (Devereux, J., et al., Nucleic Acids Research, 12(1):387 (1984)), BLASTP, BLASTN and FASTA (Altschul, S. F. et al., J. Molec. Biol., 215:403-410 (1990). The BLASTX program is publicly available from NCBI and other sources (BLAST Manual, Altschul, S. et al., NCVI NLM NIH Bethesda, Md. 20894, Altschul, S. F. et al., J. Molec. Biol., 215:403-410 (1990), the teachings of which are incorporated herein by reference). These programs optimally align sequences using default gap weights in order to produce the highest level of sequence identity between the given and reference sequences. As an illustration, by a polynucleotide having a nucleotide sequence having at least, for example, 85%, preferably 90%, even more preferably 95% “sequence identity” to a reference nucleotide sequence, it is intended that the nucleotide sequence of the given polynucleotide is identical to the reference sequence except that the given polynucleotide sequence may include up to 15, preferably up to 10, even more preferably up to 5 point mutations per each 100 nucleotides of the reference nucleotide sequence. In other words, in a polynucleotide having a nucleotide sequence having at least 85%, preferably 90%, even more preferably 95% identity relative to the reference nucleotide sequence, up to 15%, preferably 10%, even more preferably 5% of the nucleotides in the reference sequence may be deleted or substituted with another nucleotide, or a number of nucleotides up to 15%, preferably 10%, even more preferably 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more consecutive groups within the reference sequence. Analogously, by a polypeptide having a given amino acid sequence having at least, for example, 85%, preferably 90%, even more preferably 95% sequence identity to a reference amino acid sequence, it is intended that the given amino acid sequence of the polypeptide is identical to the reference sequence except that the given polypeptide sequence may include up to 15, preferably up to 10, even more preferably up to 5 amino acid alterations per each 100 amino acids of the reference amino acid sequence. In other words, to obtain a given polypeptide sequence having at least 85%, preferably 90%, even more preferably 95% sequence identity with a reference amino acid sequence, up to 15%, preferably up to 10%, even more preferably up to 5% of the amino acid residues in the reference sequence may be deleted or substituted with another amino acid, or a number of amino acids up to 15%, preferably up to 10%, even more preferably up to 5% of the total number of amino acid residues in the reference sequence may be inserted into the reference sequence. These alterations of the reference sequence may occur at the amino or the carboxy terminal positions of the reference amino acid sequence or anywhere between those terminal positions, interspersed either individually among residues in the reference sequence or in the one or more consecutive groups within the reference sequence. Preferably, residue positions which are not identical differ by conservative amino acid substitutions. However, conservative substitutions are not included as a match when determining sequence identity.

“Sequence homology”, as used herein, refers to a method of determining the relatedness of two sequences. To determine sequence homology, two or more sequences are optimally aligned, and gaps are introduced if necessary. However, in contrast to “sequence identity”, conservative amino acid substitutions are counted as a match when determining sequence homology. In other words, to obtain a polypeptide or polynucleotide having 95% sequence homology with a reference sequence, 85%, preferably 90%, even more preferably 95% of the amino acid residues or nucleotides in the reference sequence must match or comprise a conservative substitution with another amino acid or nucleotide, or a number of amino acids or nucleotides up to 15%, preferably up to 10%, even more preferably up to 5% of the total amino acid residues or nucleotides, not including conservative substitutions, in the reference sequence may be inserted into the reference sequence.

“Isolated” means altered “by the hand of man” from its natural state, i.e., if it occurs in nature, it has been changed or removed from its original environment, or both. For example, a polynucleotide or polypeptide naturally present in a living organism is not “isolated,” but the same polynucleotide or polypeptide separated from the coexisting materials of its natural state is “isolated”, as the term is employed herein.

As used herein, “a pharmaceutically-acceptable carrier” includes any and all solvents, dispersion media, coatings, adjuvants, stabilizing agents, diluents, preservatives, antibacterial and antifungal agents, isotonic agents, adsorption delaying agents, and the like. In addition, the immunogenic and vaccine compositions of the present invention can include diluents, isotonic agents, stabilizers, or adjuvants. Diluents can include water, saline, dextrose, ethanol, glycerol, and the like. Isotonic agents can include sodium chloride, dextrose, mannitol, sorbitol, and lactose, among others. Stabilizers include albumin and alkali salts of ethylendiamintetracetic acid, among others

### EXAMPLE

The following example sets forth preferred embodiments of the present invention. These embodiments demonstrate a method for inducing EEAU. However, these embodiments are for illustrative purposes only and the disclosure herein should not be construed as a limitation upon the scope of the present invention.

### Example 1

This example illustrates a method for inducing EAAU by MAA-derived peptides.

Materials and Methods

Peptides having the sequence of SEQ ID NO. 1 and SEQ ID NO. 2 were solubilized in sterile phosphate buffered saline (PBS) before use. Lewis rats were immunized separately with 100 μl of stable emulsion containing 250 μg of peptide SEQ ID NO. 1 (n=9 rats) or peptide SEQ ID NO. 2 (n=9 rats) emulsified (1:1) in complete Freund's adjuvant (CFA) using a single-dose induction protocol in the hind footpad to induce EAAU. Control animals (n=9 rats) received 75 μg of bovine melanin associated antigen (MAA) emulsified (1:1) in CFA. Animals in each group were examined daily between day 7 and 30, post-immunization, to determine the time of onset and progression of clinical disease in EAAU. Two animals from each group were sacrificed at day 17 and day 20 post-immunization and eyes were harvested for histological analysis to determine the severity of disease and degree of inflammation. Lewis rats injected with MAA peptide SEQ ID NO. 1 or MAA peptide SEQ ID NO. 2 developed EAAU. The clinical and histopathologic examination revealed that EAAU induced by peptide SEQ ID NO. 1 (FIGS. 1A and 1D) and peptide SEQ ID NO. 2 (FIGS. 1B and 1E) was less severe than that induced by bovine MAA (FIGS. 1C and 1F). As shown in all of the Figs., the iris (I) and the ciliary body (CB) were infiltrated with inflammatory cells.

What is claimed is:

1. An immunogenic composition, the composition comprising: a truncated peptide consisting of a peptide with at least 90% homology to at least 15 contiguous amino acids selected from amino acids 101 to 150 of SEQ ID NO:3.

2. The immunogenic composition of claim 1, wherein said truncated peptide has a length of about 25 amino acids.

3. The immunogenic composition of claim 1, wherein said truncated peptide is selected from the group consisting of SEQ ID NOS 1 and 2.

4. The immunogenic composition of claim 1, further comprising a pharmaceutically-acceptable carrier selected from the group consisting of solvents, dispersion media, coatings, adjuvants, stabilizing agents, diluents, preservatives, antibacterial and antifungal agents, isotonic agents, adsorption delaying agents, and combinations thereof.

5. The immunogenic composition of claim 4, wherein said pharmaceutically acceptable carrier includes an adjuvant, and wherein said adjuvant if Freund's complete adjuvant.

6. The immunogenic composition of claim 4, wherein said pharmaceutically acceptable carrier includes phosphate buffered saline.

7. A method of inducing experimental autoimmune anterior uveitis comprising the step of administering the immunogenic composition of claim 1 to a subject.

8. The method of claim 7, wherein said administering step is performed using an administration route selected from the group consisting of oral, nasal, intravenous, intramuscular, intraocular, topical, subcutaneous, and combinations thereof.

9. The method of claim 7, wherein said immunogenic composition further comprises a pharmaceutically-acceptable carrier selected from the group consisting of solvents, dispersion media, coatings, adjuvants, stabilizing agents, diluents, preservatives, antibacterial and antifungal agents, isotonic agents, adsorption delaying agents, and combinations thereof.

10. The method of claim 7, wherein said subject is selected from the group consisting of rats and humans.

11. The method of claim 7, wherein said administering step is repeated.

12. The method of claim 7, wherein 10 μg to 1500 μg of said peptide is present in each administration of said immunogenic composition.

13. The method of claim 12, wherein 75 μg to 400 μg of said peptide is present in each administration of said immunogenic composition.

14. A method of treating anterior uveitis comprising the step of administering the immunogenic composition of claim 1 to a subject in need thereof.

15. The method of claim 14, wherein said administering step is performed using an administration route selected from the group consisting of oral, nasal, intravenous, intramuscular, intraocular, topical, subcutaneous, and combinations thereof.

16. The method of claim 14, wherein said immunogenic composition further comprises a pharmaceutically-acceptable carrier selected from the group consisting of solvents, dispersion media, coatings, adjuvants, stabilizing agents, diluents, preservatives, antibacterial and antifungal agents, isotonic agents, adsorption delaying agents, and combinations thereof.

17. The method of claim 14, wherein said subject is selected from the group consisting of rats and humans.

18. The method of claim 14, wherein said administering step is repeated.

19. The method of claim 14, wherein 10 μg to 1500 μg of said peptide is present in each administration of said immunogenic composition.

20. The method of claim 19, wherein 75 μg to 400 μg of said peptide is present in each administration of said immunogenic composition


SEQUENCE LISTING (3 sequences)

SEQ ID NO: 1 - protein, 25 aa, Bos taurus
GEPGNIGFPGPKGPSGDPGKAGEKG

SEQ ID NO: 2 - protein, 25 aa, Bos taurus
HAGLAGARGAPGPDGNNGAQGPPGL

SEQ ID NO: 3 - protein, 200 aa, Bos taurus
IGPAGPPGPPGLRGNPGSRGLPGADGRAGVMGPAGSRGATGPAGVRGPNGDSGRPGEPGLMGPRGFPGSPGNIGPAGKEGPVGLPGIDGRPGPIGPAGARGEPGNIGFPGPKGPSGDPGKAGEKGHAGLAGARGAPGPDGNNGAQGPPGLQGVQGGKGEQGPAGPPGFQGLPGPAGTAGEAGKPGERGIPGEFGLPGPAG